(19) United States
(12) Patent Application Publication
(10) Pub. No.: US 2025/0384393 A1
(43) Pub. Date: Dec. 18, 2025

(54) PROCESS FOR DISTRIBUTION, INVENTORYING, INVOICING, AND USE OF MEDICAL DEVICES

(71) Applicant: ORTHOFUNDAMENTALS, LLC, Waltham, MA (US)

(72) Inventors: Matthew Palmer, Weston, MA (US); Jarred Sakakeeny, Hopkinton, MA (US)

(21) Appl. No.: 18/879,875

(22) PCT Filed: Jun. 30, 2023
(86) PCT No.: PCT/US2023/026808

§ 371 Date: Dec. 30, 2024

Related U.S. Application Data
(60) Provisional application No. 63/357,646, filed on Jul. 1, 2022.

(51) Int. Cl. G06Q 10/087; G06K 7/10; G06K 7/14; G16H 40/20

(52) U.S. Cl. CPC G06Q 10/08726; G06K 7/10366; G06K 7/1417; G16H 40/20

(57) ABSTRACT

Systems and methods for real-time supply chain management pertaining to medical devices. The distribution, inventorying, invoicing and use of medical devices may be facilitated. The medical devices may be implants and/or instruments, such as those for use in orthopedic procedures.

### CROSS-REFERENCE TO RELATED APPLICATION

This application claims the benefit of priority to U.S. Provisional Patent Application Ser. No. 63/357,646, filed on Jul. 1, 2022 and titled PROCESS FOR DISTRIBUTION, INVENTORYING, INVOICING, AND USE OF MEDICAL DEVICES, the entire disclosure of which is hereby incorporated herein by reference in its entirety for all purposes.

### FIELD OF THE INVENTION

The present invention relates to equipment, process and methods for distributing, inventorying, invoicing, and using medical devices. The invention finds particular utility in the ability to track inventory levels, identify when a device was used, bill for the device, and restock the device so that the healthcare facility always has a full set of inventory. While the invention has applications for many different medical devices, its utility will be illustrated in the context of orthopedic implants and instruments, and more specifically for sacroiliac joint fusion implants and instruments.

### BACKGROUND

The present invention relates generally to supply chain management, including the distribution, invoicing, tracking, transporting, storage, and use of medical devices. Specifically, in the field of orthopedic surgery, many of these responsibilities are overseen by sales representatives. This is costly and because of the human element is prone to mistakes and missed steps. Typically, when a surgery is scheduled, a sales representative is responsible for finding the inventory needed for the upcoming surgery, checking it into the hospital so that it can be sterilized, bringing it from sterile storage to the operating room, logging the components of the kit that were used, invoicing the hospital or surgery center, and finally replenishing the inventory that was used.

Thus, there remains a need for inventory management systems that can efficiently and accurately automate many of the tasks performed by the sales representative.

### SUMMARY

In one embodiment of the present invention, a medical device transportation and storage device is provided. Said transportation and storage device is equipped with an RFID reader, wireless internet or cellular connection, and may also include GPS logging. Sterile packed implants and single use disposable instruments are packed within the transportation and storage device. Each implant and instrument box is tagged with a unique RFID tag as well as a QR code. The RFID tag and QR code identify the implant and/or instrument (lot number, part number, expiration date, etc.). The transportation and storage device can be shipped to a hospital or surgery center and stored there. The RFID scanner scans the contents of the transportation and storage device periodically, when the device is opened and closed, and on a pre-determined schedule. Thus the system can tell when inventory is taken from the device, by noting a missing RFID tag. The missing RFID tags may be associated with implants and instruments that were used in a surgical case, and the transportation and storage device can thus create an invoice for the used supplies.

Additionally, a mobile website/app is included that scans the QR code printed on the implant and instrument boxes. The mobile website/app may be accessed from a phone, or may be accessed using monitor integrated into the transportation and storage device. It is common for a nurse or sales rep to log the implants and instruments that are used manually and submit them as an invoice to the hospital. The present invention includes said mobile website/app to automate this process and send an invoice electronically to a predetermined hospital or surgery center contact. Additionally, this process acts as a check on the RFID scanner and to ensure that inventory that was taken out of the transportation and storage device was used in surgery.

The company providing this inventory management system knows in real time when instruments and implants were used. With the GPS logging of the device, the company knows the location of the device where the inventory was used. Thus, the company can ship replenishment inventory directly to the hospital or surgery center. The company can also timely bill the used inventory to the hospital or surgery center.

The data stored in the RFID and QR code (lot number, part number, expiration date, etc.) allows the company to keep track of where specific inventory is, and if there is a need for a product recall, ensure that the appropriate inventory is recalled. Additionally, by linking the data stored in the RFID and QR code to a specific surgery, it is possible to know specific implants and instruments that were used in a surgery. Additionally, as the implants and instruments stored in the transportation and storage device may be sterile packaged, they have expiration dates. The company can thus ensure that products are removed from the field before they expire, or they can be moved from one location to another that will use them before they expire.

In a specific embodiment of the invention, the implants and instruments are screws and instruments for performing sacroiliac joint fusion. In this specific embodiment, multiple screw sizes (diameter and length) are used when performing a sacroiliac joint fusion. Additionally, one or more instrument kits are used. The instrument kits may include one or more guide wires or k-wires, exchange pins, a tissue dilator and screw length sizing guide, one or more drill bits, a drill guide, a screw driver, and a drive handle or ratchet. Additionally, a Jamshidi or other biopsy needle and one or more parallel guides may be included. With all the instruments and implants having RFID tags, the transportation and storage device allows the company, hospital, and/or surgery center to know if there is sufficient inventory for performing a surgical case in advance of the surgery.

In another embodiment of the invention, the implants and instruments are cages, plates, screws, and instruments for performing anterior cervical discectomy and fusion.

The disclosure contemplates all combinations of any one or more of the foregoing aspects and/or embodiments, as well as combinations with any one or more of the embodiments set forth in the detailed description and any examples.

### BRIEF DESCRIPTION OF THE DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1 is a schematic showing the process workflow associated with the supply chain management, including the distribution, invoicing, tracking, transporting, storage, and use, of medical devices; and

FIGS. 2-4 present schematics of a transportation and storage device, as well as contents thereof, in accordance with various embodiments.

### DETAILED DESCRIPTION

In accordance with one or more embodiments, the efficiency, accuracy and data transparency pertaining to the operation of various healthcare facilities may be augmented. It is often unclear precisely which devices and/or instruments (i.e. size or type) will be required until a specific medical procedure is underway which, in turn, poses related inventory challenges. The readiness and logistics around any required devices and/or instruments associated with a procedure may beneficially be ensured. In some embodiments, a healthcare facility may be a hospital or surgical center. In at least some embodiments, the healthcare facility may be an orthopedic care center. Scan data and/or location data in connection with a transportation and storage device may facilitate supply chain management of medical devices. Turnaround time, understock or overstock may be reduced via real-time monitoring. Billing delays may also be reduced. Records and reporting may be facilitated. Real-time use and contents tracking may facilitate inventory management as well as the accuracy and efficiency of related logistics. Data discrepancies with respect to inventory, location, use and invoicing can be identified, evaluated and reconciled.

In accordance with one or more embodiments, a supply chain management system for medical devices, e.g. implants and/or instruments is disclosed. In some specific embodiments, the system may find particular use in the field of orthopedics. In at least some embodiments, the system is for use at an orthopedic healthcare facility to facilitate various procedures including but not limited to sacroiliac joint fusion.

In accordance with one or more embodiments, the system may include a transportation and storage device for holding various implants and/or instruments. The device may include a main body and a door movable open/closed relative to the main body. The main body and door may cooperate to provide an interior compartment for holding various implants and/or instruments. The contents may be individually packaged as further described herein. The device may be stocked by a manufacturer and then shipped to an end-use hospital or surgery center for onsite use. The device may include location technology (e.g. GPS). The device may be enabled with radio frequency identification (RFID) technology to maintain automated and “real time” inventory levels of the items that are inside the device. The transportation and storage device may include various environmental sensors (interior/exterior) including but not limited to door sensor (open/closed), temperature, pressure, humidity, impact and/or acceleration. The container may include a controller in communication with RFID scanner/detector and any sensors to gather and transmit associated data to a remote device over a network. A communication interface (e.g. wireless) may facilitate transmission. A screen may be included to provide visual access to inventory summaries and to aid in the invoicing of implants and instruments. The remote device may be a mobile device/app, personal computer, enterprise resource planning system, website, server, etc. Such communication or reporting can occur continuously, at predetermined time intervals, or responsive to an event or condition. The remote device may use data received to automatically update and generate appropriate records pertaining to inventory. The device may optionally be equipped to provide one or more associated indicators. A power source may be onboard the device and the control system may optionally be configured to run in a periodic sleep state. One or more security or access limiting/tracking features (e.g lock, badge/card/fingerprint reader, etc.) may be included.

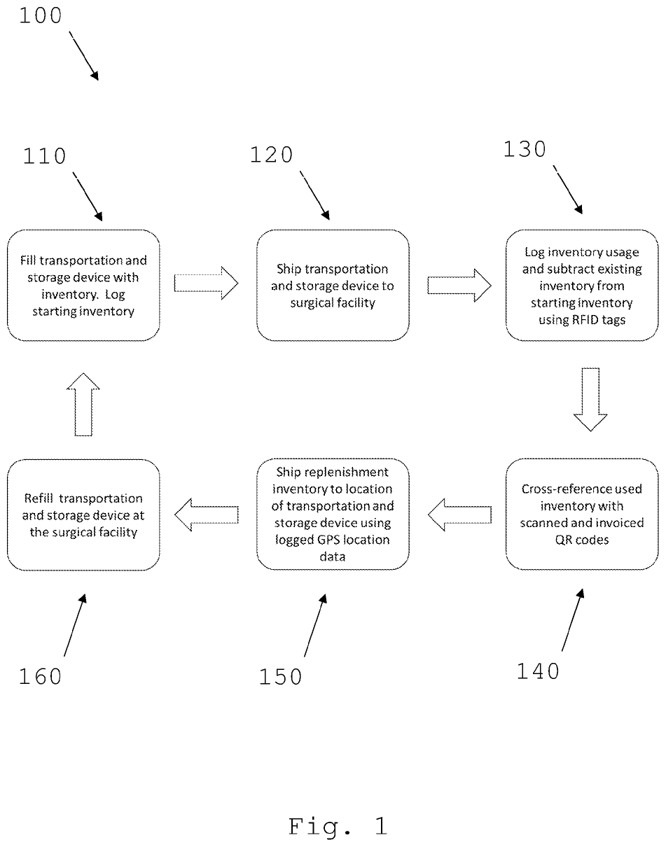

Looking at FIG. 1 there is shown a process workflow, 100, associated with the disclosed supply chain management system, including the distribution, invoicing, tracking, transporting, storage, and use, of medical devices.

In a first step, 110, the company assembles the transportation and storage device with sterile packed instruments and implants. The implants and instruments are placed inside sterile barriers. The sterile product is labeled and placed in individual boxes. The boxes are labeled. The label includes a QR code. The QR code includes product information including part number, lot number, and expiration date. Each box also is labeled with an RFID tag. The RFID tag is linked to a specific part number, lot number, and expiration date.

Additionally, the transportation and storage device includes a RFID reader, internet connectivity (wired or wireless, WIFI or cellular), and GPS position monitoring. The RFID reader can be programmed to scan the contents of the transportation and storage device on demand, whenever the device is opened or closed, and/or at predetermined times. Thus, the company always knows what the contents of the device are (by scanning the QR codes) and where the device is (by reading GPS location).

Looking now at 120, the contents of the transportation and storage device are logged prior to shipping the device to the hospital or surgery center that will be using the implants and instruments. GPS location data can notify the company when the device has arrived at its desired location.

Looking now at 130, the contents of the device are logged periodically. By subtracting the current RFID tag contents from the starting contents, the company is notified of any devices that were removed from the transportation and storage device. The contents that were removed can be assumed to have been used in a surgery case. As an example, the contents of the case may be taken out of the transportation and storage device and brought into the operating room. Following the procedure, the contents are returned to the device. The device can then notify the company what instruments and implants were used. These can then be billed to the hospital or surgery center and can be linked to a specific surgery.

Looking now at 140 a separate website/mobile app can be used by a sales representative or healthcare facility employee to log the QR codes printed on the implants and instruments that were used. Additionally, the healthcare facility location, patient information, and surgery date can be inputted into the website/mobile app. When this information is submitted, an invoice is generated and electronically sent to a contact at the healthcare facility and is also sent to the company. The company can thus check to ensure that the missing RFID tags are associated with the QR codes that were tagged. If they do not match, the healthcare facility can be contacted to assist with locating the missing devices.

It is possible that step 140 is not required if the transportation and storage device allows the user to input healthcare facility location, patient information, and surgery date and link the implants and instruments to the missing RFID tags. In this case, QR codes may be used as a back up.

Looking now at 150, the company can ship replenishments for the implants and instruments that were used at the healthcare facility. The company can cross reference GPS data from the transportation and storage device with the invoice location to ensure replenishments are shipped to the correct location.

Finally, and now looking at 160, a sales representative or healthcare facility employee can receive the replenishment inventory and restock the transportation and storage device. The RFID tags can be scanned and added to the existing device inventory.

In a specific embodiment of the invention the implants are sterile screws for sacroiliac joint fusion, and the instruments are sterile single use instruments for performing sacral iliac joint fusion.

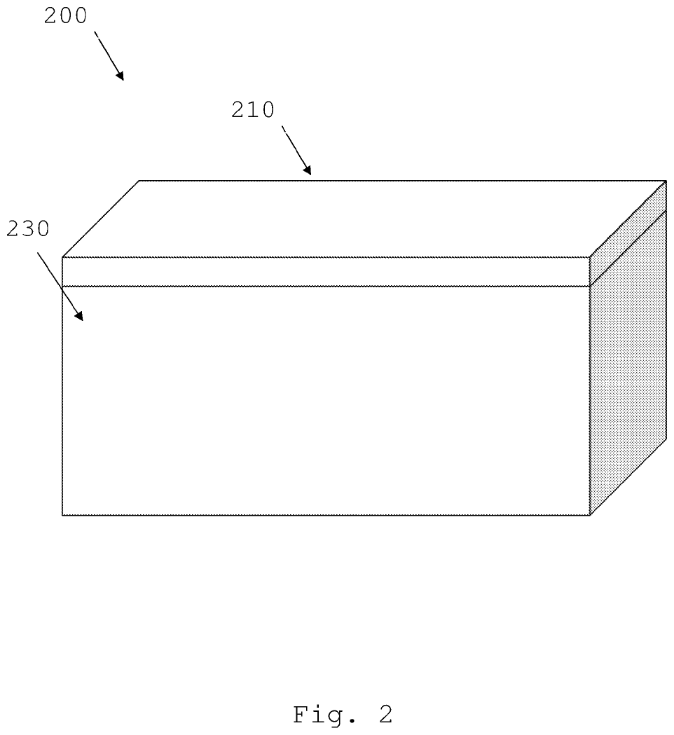

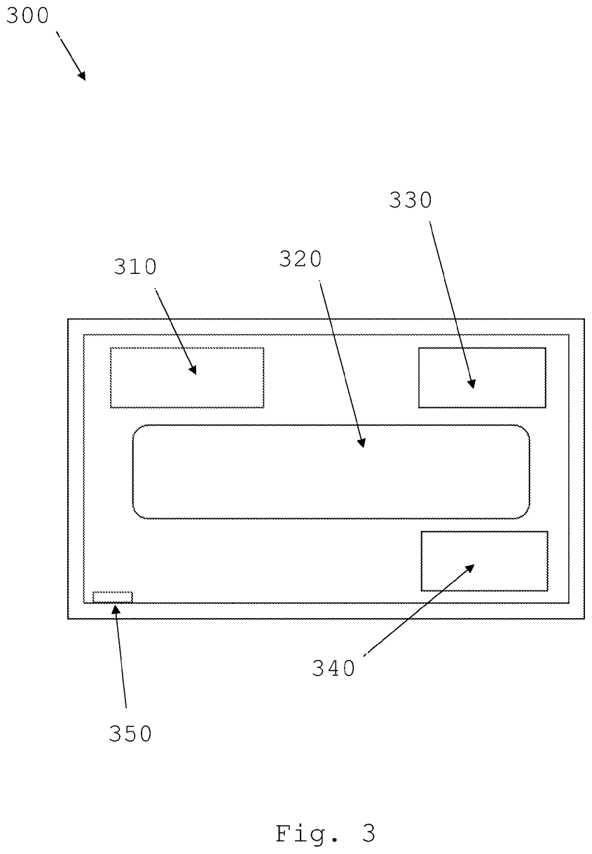

Looking now at FIG. 2, a representative model of the transportation and storage device 200 are shown. The case 200 has a lid 210 and a main body 230 for storing inventory. Looking into the main body 230, there may be a false bottom, and under the false bottom, the electronic components of the transportation and storage device are shown. FIG. 3 shows the electronic components, 300. The electronic components include RFID reader 310, RFID antennae 320, a microcontroller 330, a battery 340, and a switch 350, that can sense when the cases is opened and closed. Inventory can sit above this false bottom.

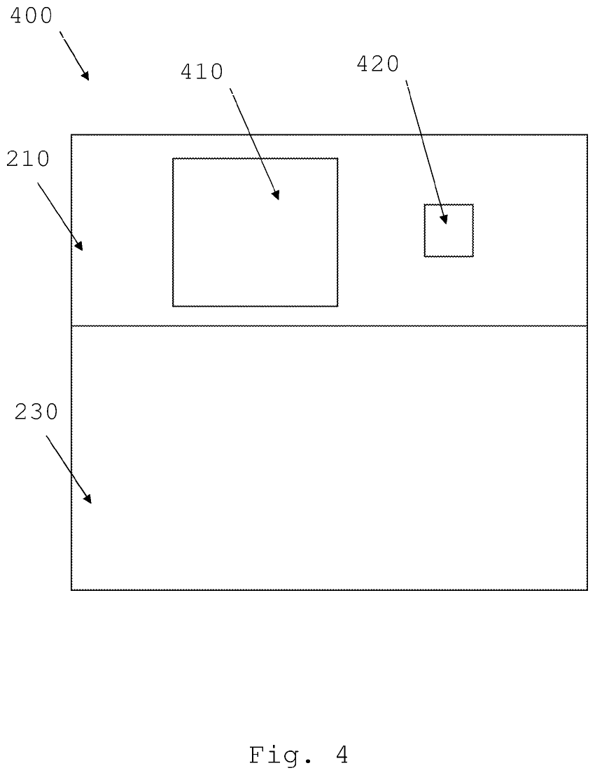

Looking now at FIG. 4 the transportation and storage device is shown from the front with the lid 210 open. Inside the lid 210, a monitor 410 is shown. Monitor 410 may be a touch screen and may be used to display available inventory and used inventory. Additionally, it may be used to input additional information into a log of used inventory to tie the used inventory to a specific surgery. A separate barcode scanner 420 may also be incorporated. Barcode scanner 420 may be a camera or other barcode scanner and can be used to scan the QR codes on the implant and instrument boxes.

Screen 410 may also be used to display additional information on the product and surgical technique guides and instructions for use. In cases when barcode scanner 420 is a camera, the camera can be used with the monitor to teleconference/video conference with the company or other technical support. If barcode scanner 420 is not a camera, then a separate camera may be used.

The transportation and storage device enables automatic billing and replenishment of consumed inventory. In cases where the inventory is provided sterile packed, a sales rep is not needed to drop off inventory before a case, pick up the inventory after the case, and re-supply the case with inventory. The replenishing of inventory is automated and allows the sales rep to spend more time selling.

### Modifications of the Preferred Embodiments

It should be understood that many additional changes in the details, materials, steps and arrangements of parts, which have been herein described and illustrated in order to explain the nature of the present invention, may be made by those skilled in the art while still remaining within the principles and scope of the invention.

The phraseology and terminology used herein is for the purpose of description and should not be regarded as limiting. As used herein, the term “plurality” refers to two or more items or components. The terms “comprising,” “including,” “carrying,” “having,” “containing,” and “involving,” whether in the written description or the claims and the like, are open-ended terms, i.e., to mean “including but not limited to.” Thus, the use of such terms is meant to encompass the items listed thereafter, and equivalents thereof, as well as additional items. Only the transitional phrases “consisting of” and “consisting essentially of,” are closed or semi-closed transitional phrases, respectively, with respect to the claims. Use of ordinal terms such as “first,” “second,” “third,” and the like in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

Having thus described several aspects of at least one embodiment, it is to be appreciated various alterations, modifications, and improvements will readily occur to those skilled in the art. Any feature described in any embodiment may be included in or substituted for any feature of any other embodiment. Such alterations, modifications, and improvements are intended to be part of this disclosure and are intended to be within the scope of the invention. Accordingly, the foregoing description and drawings are by way of example only.

Those skilled in the art should appreciate that the parameters and configurations described herein are exemplary and that actual parameters and/or configurations will depend on the specific application in which the disclosed methods and materials are used. Those skilled in the art should also recognize or be able to ascertain, using no more than routine experimentation, equivalents to the specific embodiments disclosed.

1. A supply chain management system for medical devices that utilizes a transportation and storage device, RFID tags, a mobile RFID scanner, position monitoring, QR codes, and a mobile app to:
transport medical devices to a healthcare facility;
log inventory usage using RFID data;
ship replenishment devices to the healthcare facility; and
restock the transportation and storage device.

2. A supply chain management system according to claim 1 wherein there is an additional step of cross-referencing inventory usage using QR codes and a mobile app.

3. A supply chain management system according to claim 1 wherein the medical devices are sterile packed screws for sacroiliac joint fusion.

4. A supply chain management system according to claim 1 wherein the medical devices are sterile single use instruments for performing sacroiliac joint fusions.

5. A supply chain management system according to claim 1 wherein the RFID tags of the transportation and storage device are scanned every time the case is opened and closed.

6. A supply chain management system according to claim 1 wherein the RFID tags of the transportation and storage device are scanned at predetermined time intervals.

7. A supply chain management system according to claim 1 wherein an initial inventory of the transportation and storage device is established as a baseline prior to shipment to the healthcare facility.

8. A supply chain management system according to claim 1 wherein a sales representative or employee can tag QR codes to log specific inventory used in a surgery case.

9. A supply chain management system according to claim 8 wherein missing RFID tags may be cross-referenced with tagged QR codes to verify specific inventory used during the surgery case.

10. A supply chain management system according to claim 1 wherein an invoice may be generated upon onsite comparison of the initial inventory to a subsequent inventory.

11. A supply chain management system according to claim 1 wherein replenishment inventory is shipped in real-time directly to the healthcare facility rather than returning the transportation and storage device to a manufacturer or a distribution center for refill.

12. A supply chain management system according to claim 11 wherein GPS data is cross-referenced to ensure replenishment inventory is directed to the correct location.

13. A supply chain management system according to claim 11 wherein inventory replenishment associated with the transportation and storage device is performed locally by a sales representative or employee affiliated with the healthcare facility by adding scanned RFID tags to existing inventory of the transportation and storage device.
